(19) United States
(12) Patent Application Publication
(10) Pub. No.: US 2026/0358324 A1
(43) Pub. Date: Nov. 12, 2026

(54) METHOD FOR DETERMINING ASPHALTENE FLOCCULATION CONDITIONS IN A HYDROCARBON FLUID

(71) Applicant: IFP Energies nouvelles, RUEIL-MALMAISON (FR)

(72) Inventors: Nicolas FERRANDO, RUEIL-MALMAISON CEDEX (FR); Benoit CRETON, RUEIL-MALMAISON CEDEX (FR); Saheb MAGHSOODLOO, RUEIL-MALMAISON CEDEX (FR); Pascal MOUGIN, RUEIL-MALMAISON CEDEX (FR); Tri Dat NGO, RUEIL-MALMAISON CEDEX (FR)

(21) Appl. No.: 19/129,843

(22) PCT Filed: Nov. 7, 2023
(86) PCT No.: PCT/EP2023/080908

§ 371 Date: May 14, 2025

(30) Foreign Application Priority Data
Nov. 21, 2022 (FR) FR2212102

(51) Int. Cl. G01N 33/28; G06F 30/27; G06F 30/28

(52) U.S. Cl. CPC G01N 33/2835; G06F 30/27; G06F 30/28

(57) ABSTRACT

The present invention relates to a process for determination of flocculation conditions of a hydrocarbon fluid in which a flocculation model is applied to the hydrocarbon fluid, and also to a compositional model. Subsequently, flocculation parameters obtained by the associated compositional model are compared with a thermodynamic model and the flocculation model to calibrate the compositional model.

### CROSS-REFERENCE TO RELATED APPLICATIONS

Reference is made to PCT/EP2023/080908 filed Nov. 7, 2023, and French Application No. 2212102, filed Nov. 21, 2022, which are incorporated herein by reference in their entirety.

### BACKGROUND OF THE INVENTION

### Field of the Invention

The present invention relates to the field of the characterization of a hydrocarbon fluid, with a view to optimizing its exploitation.

### Description of the Prior Art

Asphaltenes are the heaviest and most polar components of a petroleum cut. This is because a hydrocarbon fluid, also called a petroleum fluid, is a continuous series of hydrocarbons (alkanes, naphthenes, aromatics). Asphaltenes are defined, according to the French standard AFNOR T 60-115, as the fraction of a petroleum fluid which flocculates (precipitates) in n-heptane and which is soluble in benzene. It thus concerns a solubility class. These asphaltenes flocculate under the effect of three factors: variation in temperature, pressure or chemical composition. This is because the modification of the composition of the fluid can also induce the phenomenon of flocculation. The addition of light alkanes will produce flocculation of the asphaltenes whereas the addition of aromatics will make it possible to stabilize them in the crude.

During the production of a well, the hydrocarbon fluid undergoes variations in pressure, in temperature and in composition. It is thus liable to flocculate, that is to say to form an aggregate of solid particles. This flocculation will be reflected by a deposition of asphaltenes in the pores of the reservoir, the consequence of which is a modification in the porosity of the medium, in its permeability and finally in the production of hydrocarbons. In the ultimate cases, this flocculation can induce clogging of the pores and bring about the shutdown of the well. It is thus important to be able to predict this phenomenon.

Furthermore, asphaltene flocculation can represent a risk of blocking of wells and production lines. During the exploitation of asphaltenic petroleum fluids, it is thus fundamental to evaluate the risk of flocculation.

This evaluation is based on the determination of the flocculation curve of the fluid, which represents the amount of flocculated asphaltene as a function of the pressure for a given temperature (see example FIG. 1). FIG. 1 shows a curve of the amount of precipitated asphaltene w as a function of pressure P for a given temperature. This curve is called the flocculation curve. Starting from the low pressure, flocculation starts from a limiting pressure called the lower threshold pressure (PL). The maximum of flocculated asphaltene wₘₐₓ is reached near the saturation pressure of the fluid (Pₛₐₜ). On continuing to increase the pressure, the amount of flocculated asphaltene subsequently decreases, until it disappears at the upper threshold pressure (P_{U}). Flocculation is at a maximum (and the amount of asphaltenes in the liquid at a minimum) when the bubble pressure (appearance of the first vapor bubble) is reached. Beyond this pressure, the appearance of a vapor phase rich in alkanes, and thus in flocculating products, makes it possible for the liquid to be again receptive with regard to the asphaltenes.

The flocculation curve of a hydrocarbon fluid can be determined by use of laboratory experiments but these experiments can be lengthy and costly. The use of predictive models to determine this curve is thus an advantageous alternative, especially for software dedicated to reservoir simulation and to the transportation of petroleum fluids.

The patent application FR 2 836 719 (US 2003/0167157) describes a method for predicting a flocculation curve of a petroleum fluid from homothetic transformations of the flocculation curve of a reference fluid. The flocculation curve of the reference fluid is obtained beforehand by experimental measurements. This method thus requires experimental data in order to have a flocculation curve.

Sanchez, N. L., A General Approach for Asphaltene Modeling, SPE-107191-MS, Latin American & Caribbean Petroleum Engineering Conference, Buenos Aires, Argentina, April 2007, describes a method for determining a flocculation curve. This method employs a decomposition of the fluid into seven constituents and a calibration of the composition of the heavy constituent based on experimental flocculation data. This method requires experimental data in order to have a flocculation curve.

Nghiem, L. X., Khose, B. F. and Farouq Ali, S. M., Asphaltene Precipitation: Phase Behaviour Modelling and Compositional Simulation, SPE-59432, Asia Pacific Conference on Integrated Modelling for Asset Management, Yokohama, Japan, April 2000, describes a method for determining a flocculation curve. This method proposes to separate the heavy constituent of the fluid into two fractions, and to treat the asphaltene phase as a solid phase. The calibration of the fluid concerns parameters of binary interaction between the heavy and light constituents, based on experimental flocculation data. This method requires experimental data in order to have a flocculation curve.

Szewczyk, V. and Behar, E., Compositional model for predicting asphaltenes flocculation, Fluid Phase Equilibria, 185-160 (1999), 459-469, describes a method of representation of the fluid according to a standardized representation having 33 constituents, and a calibration of the molar mass and the critical temperature of the heavy constituent on the basis of experimental flocculation data. This method requires experimental data in order to have a flocculation curve. Moreover, the models used in this representation are complex, and thus cumbersome to employ with computer systems (computer memory, processors).

### SUMMARY OF THE INVENTION

The invention determines asphaltene flocculation conditions of a hydrocarbon fluid, in a precise, simple and rapid manner, without requiring experimental measurements. For this purpose, the invention relates to a process for determination of flocculation conditions of a hydrocarbon fluid in which a flocculation model is applied to the hydrocarbon fluid, and also to a compositional model. Subsequently, flocculation parameters obtained by the associated compositional model are compared with a thermodynamic model and the flocculation model to calibrate the compositional model. The flocculation model for the fluid makes it possible to do without experimental measurements to calibrate the compositional model. Thus, it is possible to obtain the flocculation conditions in a simple and rapid manner.

The invention also relates to a process for determination or prediction of asphaltene flocculation, and also to a process for exploitation of a hydrocarbon fluid.

The invention relates to a process for determination of conditions for asphaltene flocculation of a hydrocarbon fluid. For this process, the following stages are carried out:
a) a flocculation model is applied to the hydrocarbon fluid in order to determine at least one modeled flocculation parameter, the flocculation model relating the composition of a fluid to at least one flocculation parameter;
b) a compositional model of the hydrocarbon fluid is constructed, the compositional model decomposing the hydrocarbon fluid into several constituents, and at least one physical property of the hydrocarbon fluid is determined by use of the compositional model;
c) a thermodynamic model is applied to the at least one physical property determined by use of the compositional model in order to determine at least one flocculation parameter;
d) the compositional model of the hydrocarbon fluid is calibrated by minimization of a difference between at least one flocculation parameter obtained from the thermodynamic model and the at least one modeled flocculation parameter obtained from the flocculation model; and
e) the conditions for asphaltene flocculation of the hydrocarbon fluid are determined from the calibrated compositional model of the hydrocarbon fluid.

According to one embodiment, the process comprises a preliminary stage of construction of the flocculation model by machine learning, in which the following stages are carried out:
i) a training database of training fluids is constructed, each training fluid being characterized by a composition and at least one flocculation parameter, preferably the at least one flocculation parameter for the training fluids being obtained experimentally; and
ii) a flocculation model is constructed by use of a machine learning method trained on the training database, the flocculation model relating the detailed composition of a fluid to the at least one flocculation parameter.

Advantageously, each flocculation parameter is chosen from a lower threshold pressure, an upper threshold pressure, a saturation pressure or a maximum amount of flocculated asphaltene.

Advantageously, the compositional model is based on a representation in N constituents, N being of between 6 and 15, preferably between 6 and 12, and preferably having the value 10.

According to one aspect, the compositional model is calibrated by use of an iterative method.

In accordance with one implementation, the thermodynamic model is an algorithm for calculating the three-phase liquid-liquid-vapor equilibrium of a fluid at a given temperature and at a given pressure.

According to one implementational option, the flocculation conditions correspond to a flocculation curve which relates the amount of flocculated asphaltene to the pressure of the fluid.

According to one embodiment, the at least one physical property is chosen from the molar mass, the critical temperature, the critical pressure or the acentric factor of the constituents of the fluid.

Furthermore, the invention relates to a process for determination or prediction of an asphaltene flocculation of a hydrocarbon fluid within a subterranean formation or within a fluid transportation pipe or within a conversion reactor, in which the following stages are carried out:
a) conditions for asphaltene flocculation of the hydrocarbon fluid are determined by use of the process for determination of conditions for asphaltene flocculation according to one of the preceding characteristics;
b) a pressure and a temperature of the hydrocarbon fluid within the subterranean formation or within the transportation pipe or within a conversion reactor are determined by measurement or predicted by simulation; and
c) a possible asphaltene flocculation of the hydrocarbon fluid is determined or predicted as a function of the determined flocculation conditions and of the determined or predicted pressure and of the determined or predicted temperature.

In addition, the invention relates to a process for exploitation of a hydrocarbon fluid within a subterranean formation or within a fluid transportation pipe or within a conversion reactor, in which the following stages are carried out:
d) an asphaltene flocculation of the hydrocarbon fluid is determined or predicted by use of the process for determination or prediction of a flocculation according to one of the preceding characteristics; and
e) the hydrocarbon fluid is exploited as a function of the possible asphaltene flocculation of the hydrocarbon fluid, in particular by modifying the temperature, at least one of the pressure, the composition of the hydrocarbon fluid in the case of asphaltene flocculation and by addition of solvent.

Other characteristics and advantages of the process according to the invention will become apparent on reading the description below of nonlimiting implementational examples, with reference to the appended figures described below.

### BRIEF DESCRIPTION OF THE DRAWINGS

FIG. 1, already described, illustrates an asphaltene flocculation curve.

FIG. 2 illustrates the stages of the process according to a first embodiment of the invention.

FIG. 3 illustrates the stages of the process according to a second embodiment of the invention.

FIG. 4 illustrates the stages of the process according to a third embodiment of the invention.

FIG. 5 illustrates the stages of the process according to a fourth embodiment of the invention.

FIG. 6 illustrates, for an example, a comparison between experimental pressures, pressures obtained by use of the flocculation model, and pressures determined by an embodiment of the invention.

FIG. 7 illustrates, for the example of FIG. 6, asphaltene flocculation curves determined by use of the process according to one embodiment of the invention.

### DETAILED DESCRIPTION

The present invention relates to a process for determination of conditions for asphaltene flocculation of a hydrocarbon fluid, in other words a process for determination of conditions for the flocculation of an asphaltene contained in a hydrocarbon fluid. The hydrocarbon fluid is in particular a petroleum fluid, for example oils, in particular crude oils. For example, it can concern a fluid produced by at least one of a subterranean formation, a fluid present in a petroleum fluid transportation pipe and a fluid within a conversion reactor (for example, a deasphalting reactor). The flocculation conditions are the conditions of the generation of the asphaltene flocculation. They can, for example, comprise, for a given temperature, pressure conditions (for example the lower threshold pressure, the upper threshold pressure, the saturation pressure of the fluid) and the maximum amount of flocculated asphaltene. The lower threshold pressure is the pressure from which asphaltene flocculation starts. The upper threshold pressure is the pressure from which asphaltene flocculation disappears. The saturation pressure is the pressure at which the amount of flocculated asphaltene is at a maximum.

Preferably, the asphaltene flocculation conditions can make it possible to define the flocculation curve of the hydrocarbon fluid studied for a given temperature. As a reminder, the flocculation curve relates, for a fluid, the amount of flocculated asphaltene to the pressure for a given temperature. Thus, the process according to the invention makes it possible to define characteristics related to the physics and to the chemistry of a hydrocarbon fluid.

Advantageously, it is possible to know the composition of the fluid being studied. In an alternative form, the process according to the invention can comprise a preliminary stage of determination of the composition of the fluid studied. Furthermore, it is additionally possible to know the density and the bubble pressure of the fluid studied, or other analogous characteristics.

The process according to the invention carries out the following stages:
1) application of a flocculation model;
2) construction of a compositional model;
3) application of a thermodynamic model;
4) calibration of the compositional model; and
5) determination of the asphaltene flocculation conditions.

These stages can be implemented by a computer, in particular by a computer or by a server. These stages will be described in detail in the continuation of the description.

FIG. 2 illustrates, diagrammatically and without limitation, the stages of the process according to a first embodiment. For a fluid to be studied FLU, a flocculation model FLO MOD is applied in order to determine modeled flocculation parameters. A compositional model COM MOD of this fluid FLU is also constructed. A thermodynamic model THE MOD is subsequently applied to the compositional model. The compositional model COM MOD is subsequently calibrated by means of the modeled flocculation parameters. The calibrated compositional model subsequently makes it possible to determine flocculation conditions FC.

In accordance with one embodiment of the invention, the process can comprise a preliminary stage of construction of the flocculation model by machine learning. For this embodiment, the process according to the invention can comprise the following stages:
A) construction of a training database;
B) construction of the flocculation model;
1) application of a flocculation model;
2) construction of a compositional model;
3) application of a thermodynamic model;
4) calibration of the compositional model; and
5) determination of the asphaltene flocculation conditions.

Stages B and 1 to 5 can be implemented by a computer system, in particular by a computer or by a server. Stages A and B can be carried out beforehand just once. If it is desired to apply the process according to the invention to several fluids, only stages 1 to 4 are repeated for each fluid. These stages will be described in detail in the continuation of the description. Alternatively, stages A and B can be repeated, by adding, at each iteration, to the training database, the last fluid studied. Thus, the training database is supplemented at each iteration, and the flocculation model can become more accurate.

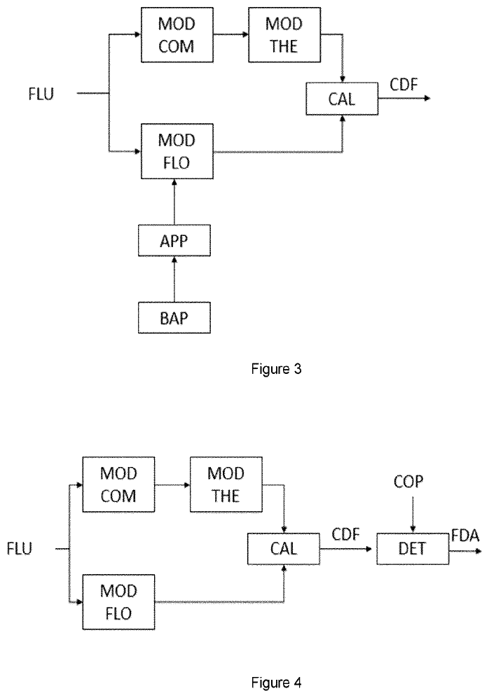

FIG. 3 illustrates, diagrammatically and without limitation, the stages of the process according to such an embodiment. The stages already described for FIG. 2 are not described in detail again. The process additionally comprises a stage of construction of a training database TDB. This training database TDB makes possible training of a machine learning method MLM in order to construct the flocculation model FLO MOD.

### A) Construction of the Training Database

During this optional stage, a training database with a plurality of training fluids is constructed. The training fluids are hydrocarbon fluids. Each training fluid is characterized by a composition and at least one flocculation parameter for a given temperature.

Each flocculation parameter can be chosen from the lower threshold pressure, denoted P_{L}, the upper threshold pressure, denoted P_{U}, the saturation pressure, denoted Pₛₐₜ, and the maximum amount of flocculated asphaltene, denoted Wₘₐₓ.

According to one embodiment, the flocculation parameters can be determined experimentally for at least one training fluid. By way of example, one of the methods described in the document: Pina et al., Oil & Gas Science and Technology-Rev. IFP, Vol. 61 (2006), No. 3, can be applied, for example, by gravimetry (cf. section 5.1), by acoustic resonance, by light scattering (cf. section 5.2) or by filtration (cf. section 5.3).

Alternatively or concurrently, the flocculation parameters can be determined for at least one training fluid from at least one published document, which discloses experimental results.

In accordance with one implementation of the invention, each training fluid can be described by a vector of several constituents (for example according to a compositional model as described in stage 2). This decomposition makes possible a good representation of each training fluid. In particular, the compositions of each training fluid can be described by a vector comprising from 8 to 33 elements, preferably from 10 to 20 elements. For (nonlimiting) example, the compositions of each fluid can be described by a vector of 14 elements according to the following distribution:
four gases: H₂S, N₂, CO₂, CH₄;
five light pseudocomponents in the C₂ to C₆ cut;
one intermediate pseudocomponent for the C₇₊ cut;
the density and the molecular weight for the C₇₊ cut; and
two heavy pseudocomponents, one per Resins and Asphaltenes fraction resulting from a SARA analysis on the C₂₀₊ cut.

The SARA analysis is a method for the characterization of heavy oils based on fractionation, in which a sample of heavy oil is separated into smaller amounts or fractions, each fraction having a different composition. The fractionation is based on the solubility of the hydrocarbon components in varied solvents. Each fraction consists of a solubility class containing a range of entities of different molecular weights. In this nonlimiting example, the crude oil is fractionated into two solubility classes, Resins and Asphaltenes, among the constituents collectively known as SARA (SARA respectively denotes: saturated hydrocarbons S, aromatics A, resins R and asphaltenes A). The saturated hydrocarbons are generally iso- and cycloparaffins, while the aromatics, the resins and the asphaltenes form a continuum of molecules with an increase in the molecular weight, in the aromaticity and in the content of heteroatoms. The asphaltenes can also contain metals, such as nickel and vanadium. This method is sometimes referred to as asphaltene/wax/hydrate deposition analysis.

According to one aspect, the training database can comprise a number of training fluids at least equal to the number of variables (the number of variables can correspond to the sum of constituents of the compositional model and of the number of physical parameters). Advantageously, the number of training fluids can comprise a number of training fluids at least equal to twice the number of variables, in order to make possible good representativeness of the different fluids and to be freed, in part, from the problem of overfitting, to ensure good reliability of the flocculation model.

### B) Construction of the Flocculation Model

During this stage, which is also optional, a flocculation model which combines at least one flocculation parameter with a fluid composition is constructed. In other words, the flocculation model makes it possible to determine at least one flocculation parameter for a hydrocarbon fluid as a function of its composition and of its temperature.

During this stage, the flocculation model is constructed by use of a machine learning method trained by use of the training database constructed as described in stage A.

During this stage, the flocculation model can be constructed by machine learning, preferably by supervised machine learning, in a preferred way by supervised regression machine learning.

According to an implementational example, the flocculation model can be written: PARAM_{f}=f (Comp, T) with f a function, PARAM_{f} a flocculation parameter (for example the lower threshold pressure, the upper threshold pressure, the saturation pressure or the maximum amount of flocculated asphaltene), T the temperature of the fluid and Comp the composition of the fluid.

The function f can be determined from a supervised learning algorithm without variable selection. The models of such an approach are linear models, insofar as each element of the vector is associated with a weighting coefficient and, for an oil, the sum of the products of the elements of the vector by their respective coefficient results in the predicted value. Alternatively, the learning algorithm can be a support vector machine (SVM), a neural network, a random forest or a combination of these methods.

For the embodiment, for which the function f can be determined from a supervised learning algorithm with variable selection, the models can be nonlinear approaches which can be obtained by Multi-Gene Genetic Programming (MGGP). Such models can be constructed by combination of elements of the vector with mathematical operators (addition, subtraction, division, product, and the like) according to a genetic evolution process (selection of models, crossing, mutation, and the like).

For both approaches, the models can be trained on one portion of the database, and tested on the other portion of the database (data external to the training process).

According to one aspect, this stage can include a validation method, preferably a cross-validation method, in particular a k-fold cross-validation method. This cross-validation method makes it possible to reduce the “overfitting” problems and to improve the accuracy of the model.

### 1) Application of a Flocculation Model

During this stage, a flocculation model is applied to the hydrocarbon fluid in order to determine at least one modeled flocculation parameter, also referred to as first flocculation parameter. The flocculation model may be known beforehand or, if appropriate, can be that constructed during the optional stages A and B. The flocculation model relates the composition of the hydrocarbon fluid to at least one flocculation parameter for a given temperature. In other words, during this stage, at least one flocculation parameter of the hydrocarbon fluid is determined by use of a flocculation model, which takes as input the composition of the fluid and the temperature. The use of such a model makes it possible to determine at least one flocculation parameter of the hydrocarbon fluid without experimentation of the hydrocarbon fluid. Thus, the determination of flocculation conditions is simpler and more rapid.

Preferably, the flocculation model can be a model trained on data (as is the case for the model constructed during the optional stages A and B). Thus, such a model does not require knowledge a priori at least one of the physical and the chemical phenomena for the determination of the flocculation parameters.

Each flocculation parameter can be chosen from the lower threshold pressure, denoted P_{L}, the upper threshold pressure, denoted P_{U}, the saturation pressure, denoted Pₛₐₜ, and the maximum amount of flocculated asphaltene, denoted Wₘₐₓ.

### 2) Construction of a Compositional Model

During this stage, a compositional model of the hydrocarbon fluid is constructed. The compositional model decomposes the hydrocarbon fluid into several constituents. Thus, such a model makes possible a simplified and structured representation of the hydrocarbon fluid, which simplifies the determination of the flocculation conditions. Preferably, the compositional model of the hydrocarbon fluid can be constructed by use of a SARA analysis, as described for stage A. In addition, during this stage, at least one physical property of the hydrocarbon fluid is determined as a function of the decomposition of the hydrocarbon fluid. This is because certain physical parameters are directly related to the composition of the fluid. Each physical property can be chosen from the molar mass, the acentric factor, the critical pressure and the critical temperature of the constituents of the hydrocarbon fluid. It is recalled that the critical temperature and the critical pressure are respectively the highest temperature and the highest pressure at which there can be a liquid-vapor equilibrium. The acentric factor is a number used in the description of matter in thermodynamics.

For the embodiment for which parameters of the constituents of the hydrocarbon fluid are known, the initial fluid can be calibrated by use of these parameters, for example the bubble pressure.

According to one embodiment of the invention, the compositional model can be based on a representation in N constituents. Advantageously, N can be of between 6 and 15. This is because a decomposition into 6 constituents makes possible a representation with a gas, a light cut and the SARA components (resulting from a SARA analysis). Thus, if fewer than 6 constituents are considered, there is a risk of the representation being less accurate. Furthermore, with a decomposition beyond 15 constituents, the accuracy is improved to the detriment of the complexity of the representation and consequently to the detriment of the complexity of calculations, of the computer time and of the computer memory used to implement the invention. Preferably, N can be of between 6 and 12, advantageously between 8 and 12, and can have the value 10. These values make possible a good compromise between accuracy of the model, and the computer time and memory which are necessary for the calculations.

For the implementational example, for which N has the value 10, the representation in 10 constituents can be as follows:
4 gases: N₂, H₂S, CO₂, CH₄;
1 light pseudocomponent in the C₂ to C₆ cut;
1 intermediate pseudocomponent in the C₇₊ to C₂₀ cut; and
4 heavy pseudocomponents, one per Saturates, Aromatics, Resins, Asphaltenes (SARA) fraction (on the C₂₀₊344 °C cut)

The SARA pseudocomponents are considered in this approach as representative of the heavy C20+ fraction, with a number of carbon atoms nC of greater than 20.

In the case where there are no heavy components with nC>20 in the initial fluid, it is considered that there is no risk of asphaltenic deposition, and it is unnecessary to implement the other stages of the process according to the invention.

A nonlimiting example of the implementation of such a compositional model with 10 constituents is described in the continuation of the description. Other approaches can be employed, for example the method described in the following document or any analogous method: Szewczyk, V. and Béhar, E., Compositional model for predicting asphaltenes flocculation, Fluid Phase Equilibria, 156-160, pp. 459-469, 1999.

The first stage of this implementation of the compositional model can be combining all the m heavy components, the number of carbons nC of which is greater than 20, within a single pseudocomponent hComp. The mean critical properties of this pseudobody are calculated via the Montel-Gouel lumping method. This stage gives an intermediate fluid denoted q1 which consists of (n−m+1) constituents: (n−m) light and intermediate components, and 1 heavy pseudocomponent hComp. It should be made clear that there is possibly a C₂₀₋ part in this pseudobody hComp: this is the case where the initial fluid given by the user does not have a C₂₀ cut and there exist some components among the m constituents having a lighter part with nC<20.

Example: an initial fluid is considered which comprises, apart from the light and intermediate components (nC<20), two heavy components, one in the C₁₂-C₂₅ cut (with nC=22) and the other in the C₂₅₊ cut (with nC=32). The objective is to convert these two components into C₁₂-C₂₀ and SARA. In the first stage, the pseudos C₁₂-C₂₅ and C₂₅+ are combined into a single pseudo C₁₂₊.

In the second stage of this implementation of the compositional model, the pseudocomponent hComp of the intermediate fluid φ₁ obtained via the previous stage is cut up in order to introduce the 4 pseudocomponents SARA. The challenge thus amounts to the decomposition of the hComp into two parts: a part C₂₀₊ corresponding to the SARA fractions and a lighter part C₂₀₋, here denoted pC₂₀₋. In the example, it is necessary to divide the pseudo C₁₂₊ into C₁₂-C₂₀ and SARA. This action is carried out based on the conservation of mass between the two representations. The physical properties (M_{w}, T_{c}, P_{c} and ω) of the mixture of 5 new pseudoconstituents have to be equivalent to those of the pseudobody hComp (C₁₂₊) which result from stage 1.

For this, an iterative approach can be used. It is a constraint optimization with 4 unknowns: the molar mass of the pseudocompound pC₂₀₋ and those of the three components Saturates, Aromatics and Resins (SAR). The molar mass of the asphaltenes is fixed at 1.0 kg/mol.

The constraints to be observed for this iterative approach can be:
Constraints of inequality: M_{w, Saturates}<M_{w, Aromatics}<M_{w, Resins}<M_{w, Asphaltenes}=1.0 kg/mol
Constraints of equality (4 constraints):

$$\begin{matrix}{\sum }_{1}^{5} {x}_{i} \times {M}_{w {,}_{i}} = {M}_{w {,}_{hComp}} where i ∈ [ {pC}_{20 -} , SARA ] \\ {\sum }_{1}^{5} {x}_{i} \times {T}_{c {,}_{i}} = {T}_{c , hComp} where i ∈ [ {pC}_{20 -} , SARA ] \\ {\sum }_{1}^{5} {x}_{i} \times {P}_{c {,}_{i}} = {P}_{c , hComp} where i ∈ [ {pC}_{20 -} , SARA ] \\ {\sum }_{1}^{5} {x}_{i} \times {ω}_{i} = {ω}_{hComp} where i ∈ [ {pC}_{20 -} , SARA ]\end{matrix}$$

with i denoting a compound of the SARA group or a pseudocompound pC₂₀, M_{w} the molar mass, T_{c} the critical temperature, P_{c} the critical pressure and x the molar fraction of the component.

In these equations, the molar fractions xi can be calculated based on a SARA analysis, and the closing condition:

$$\frac{{x}_{i} \times {M}_{w , i}}{{M}_{w , SARA}} = {wt}_{i} \% where i ∈ SARA$$

x_{pC20-}+Σ_{i∈SARA} xᵢ=1.0
   with wtᵢ the fraction by weight of SARA compounds. The limits to be observed for this iterative approach can be as follows:
M_{w,pC20-} belongs to the interval [0.045, MW_C20] where 0.045 kg/mol is the minimum molar mass valid for the pseudocomponents and MW_C20 (282.55E-3 kg/mol) corresponds to the molar mass of n-eicosane,
M_{w, Saturates}, M_{w, Aromatics}, M_{w, Resins} belong to the interval [MW_C20, 1.0].

At the end of this optimization approach, the pseudocomponent hComp is replaced by the set of new components (SARA and possibly pC₂₀₋). The intermediate fluid φ₂ thus consists of (n−m+4) or of (n−m+5) components, corresponding respectively to the case without and with pC₂₀₋.

The third stage of this implementation of the compositional model can consist in creating the asphaltene fluid of 10 components by referring to the components of the standardized representation of the target fluid by the components of the fluid φ₂. First, the gases (N₂, CO₂, H₂S, CH₄) are brought back in the final fluid if they are present in the fluid φ₂ or, failing that, zero molar fractions are identified. Subsequently, the light and intermediate hydrocarbons are classified as a function of their number of carbon atoms and combined in two pseudobodies C₂-C₇, with the number of carbon atoms which varies between 2 and 7, and C₇₊-C₂₀, with the number of carbon atoms between 7 and 20. Finally, the SARA components characterized in the second stage are brought back in the final fluid.

As regards the constitutional group, the descriptions for the gases and for the SARA fractions are already defined. The descriptions for the other two pseudobodies can be regarded as two CH₃ groups and (nCᵢ-2) CH₂ groups, where nCᵢ is is the number of carbon atoms in each pseudobody.

Subsequently, at least one physical property of the hydrocarbon fluid can be determined by use of the decomposition and of approaches based on a group contribution method. For this, it is possible to employ a conventional Montel and Gouel lumping method for the light and intermediate pseudocomponents (C₂-C₆ and C₇₊-C₂₀) and an Avaullée approach for the heavy fractions (pseudo-SARA compounds). Other approaches can be envisaged.

The conventional Montel and Gouel lumping method is described in the following document:
Montel, F. and Gouel, P. L., 1984, A New Lumping Scheme of Analytical Data for Compositional Studies, in SPE Annual Technical Conference and Exhibition, Society of Petroleum Engineers.

This method makes it possible to calculate the critical parameters, such as the critical temperature T_{c}, the critical pressure P_{c}, the molar mass M_{w} and the acentric factor ω. This method can define the following relationships:

$$\begin{matrix}{T}_{c} = \frac{( {\sum }_{i} {\sum }_{j} {x}_{i} {x}_{j} {T}_{c , i} {T}_{c , j} ) / \sqrt{{P}_{c , i} {P}_{c , j}}}{{\sum }_{i} {x}_{i} {T}_{c , i} / {P}_{c , i}} \\ {P}_{c} = \frac{{T}_{c}}{{\sum }_{i} {x}_{i} {T}_{c , i} / {P}_{c , i}} \\ {M}_{w} = {\sum }_{i} {x}_{i} {M}_{w , i} \\ ω = {\sum }_{i} {x}_{i} {ω}_{i}\end{matrix}$$

with i and j the constituents of the composition.

The Avaullée approach, also known as Avaullèe correlation, is described in the following document:
Avaullée, L., 1996, Mise au point de méthodes de caractérisation thermodynamique des fluides pétroliers en vue de la prédiction de leurs propriétés volumétriques et des expériences d′injection de gaz dans les huiles de gisement [Development of methods for thermodynamic characterization of petroleum fluids with a view to predicting their volumetric properties and gas injection experiments in formation oils], thesis of the University of Aix-Marseille, Aix-en-Provence, France, thesis number 96 AIX3.0050.

### 3) Application of a Thermodynamic Model

During this stage, a thermodynamic model is applied to the compositional model determined in stage 2. The thermodynamic model makes possible a thermodynamic balancing of the different phases of the hydrocarbon fluid, in order to determine a flocculation parameter from the compositional model. The thermodynamic model has in particular for input at least one physical property (preferably all the physical properties) resulting from the compositional model and has, for output, at least one flocculation parameter, also known as second flocculation parameter.

According to one embodiment, a thermodynamic model can carry out a thermodynamic equilibrium calculation (known as flash algorithm) capable of managing the simultaneous existence of a vapor phase, of a hydrocarbon liquid phase and of a second asphaltenic liquid phase. It can then concern a three-phase liquid-liquid-vapor equilibrium calculation. For nonlimiting example, the flash algorithm used can be that provided by Michelsen in the following documents:
Michelsen, M. L., Fluid Phase Equilibria, 9 (1982), 1-19
Michelsen M. L., Fluid Phase Equilibria, 9 (1982), 21-40.

The thermodynamic model used for this calculation in this algorithm can be the Peng-Robinson cubic equation of state coupled with the Abdoul-Péneloux mixing rule, as are described in particular in the following documents:

Abdoul, W., 1987, Une méthode de contribution de groupes applicable à la corrélation et la prédiction des propriétés thermodynamiques des fluides pétroliers [A group contribution method applicable to the correlation and the prediction of the thermodynamic properties of petroleum fluids], thesis of the University of Aix-Marseille III, France; and
Peneloux, A., Abdoul, W. and Rauzy, E., 1989, Excess Functions and Equation of State, Fluid Phase Equilibria, 47, 115-132.

Such a model can be written:

$$P = \frac{RT}{{w}_{i} - {b}_{i}} - \frac{{a}_{i} ( T )}{{w}_{i} ( {w}_{i} + γ {b}_{i} )}$$

where T and P are respectively the temperature and the pressure of the system, R is the universal perfect gas constant (R≈8.314 kPa·l/mol·K), y=4.82843 is the constant characteristic of the equation, wᵢ indicates the uncorrected molar volume, aᵢ(T) is the interaction parameter and bᵢ denotes the covolume of the component i. These parameters are calculated for the critical temperature T_{c,i}, the critical pressure P_{c,i} and the acentric factor ωᵢ which result from the compositional model. Furthermore,

$${b}_{i} = 0.04557 \times \frac{{RT}_{c , i}}{{P}_{c , i}}$$

$$and$$

$${a}_{i} ( T ) = a ( {T}_{c} ) \times α ( {T}_{r , i} , {ω}_{i} )$$

$$with \{ \begin{matrix}{T}_{r , i} = \frac{T}{{T}_{c , i}} ; a ( {T}_{c} ) = 0.45724 \times \frac{{R}^{2} {T}_{c}^{2}}{{P}_{c}} ; \\ \begin{matrix}α ( {T}_{r} , {ω}_{i} ) = {[ 1 + {m}_{i} ( 1 - {T}_{r , i}^{0.445} ) ]}^{2} ; & {m}_{i} = \frac{{( 1.12754 + 0.51725 {ω}_{i} - 0.00373 {ω}_{i}^{2} )}^{0.5} - 1}{1 - {0.7}^{0.445}} ;\end{matrix}\end{matrix}$$

The Abdoul-Peneloux approach considers the volume translation, where the “true” molar vᵢ is corrected by a term cᵢ:

$${w}_{i} = {v}_{i} + {c}_{i} = {v}_{i} + ( 0.08315 - 0.4406 {Z}_{Ra , i} ) \times \frac{{RT}_{c , i}}{{P}_{c , i}}$$

where Z_{Ra,i} is the Rackett compressibility factor. These equations are valid for pure bodies. For a mixture, the whole of the mixture can be considered as a single constituent following the same formalism. The Abdoul-Peneloux mixing rule is applied to the critical properties of the pure bodies in order to obtain the overall parameters of the mixture a, b, c.

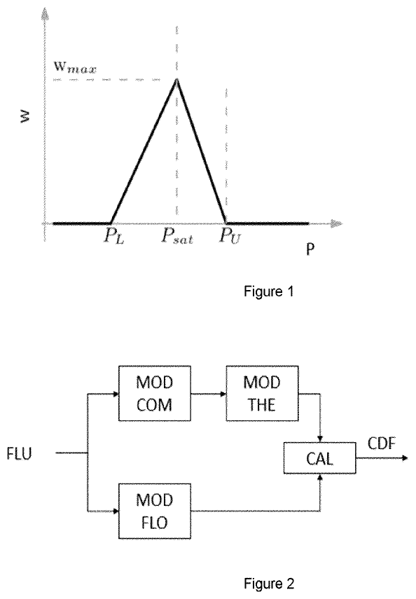

At each given temperature, a series of three-phase flash calculations (in other words, several flash algorithms are applied) can be carried out by varying the pressure in order to obtain the whole of the flocculation curve as represented diagramatically in FIG. 1. This approach makes it possible to also determine the key pressures, such as the saturation pressure Pₛₐₜ, the lower threshold pressure P_{L} and the upper threshold pressure P_{U}.

### 4) Calibration of the Compositional Model

During this stage, at least one physical property of the compositional model determined in stage 2 of the hydrocarbon fluid is calibrated by minimization of a difference between at least one flocculation parameter obtained from the thermodynamic model in stage 3 (second flocculation parameter) and the at least one modeled flocculation parameter determined in stage 1 (first flocculation parameter). In other words, the compositional model is adjusted in order for it to behave similarly to the flocculation model. This adjustment concerns the setting of at least one physical property of the compositional model in order for a flocculation parameter resulting from the compositional model to approach as close as possible to a modeled flocculation parameter resulting from the flocculation model. This calibration stage makes it possible to improve the quality of prediction of the asphaltene flocculation conditions.

According to one embodiment of the invention, the calibration can employ an iterative method, in which at least one physical property is modified at each stage until a minimum is obtained between the flocculation parameter resulting from the compositional model and the modeled flocculation parameter. For this iterative method, it is possible to seek to minimize an objective function which comprises a term measuring a difference between the calculated values of the flocculation parameters and the modeled flocculation parameters.

In accordance with one implementation of the invention, the physical properties to be calibrated can be the molar mass and the critical temperature. The other physical properties of the asphaltene constituent (for example the critical pressure and the acentric factor) can be determined as a function of the molar mass and of the critical temperature by the Avaullée approach. This approach gives a better characterization of the very heavy components in comparison with other approaches.

### 5) Determination of the Asphaltene Flocculation Conditions

During this stage, the flocculation conditions of the hydrocarbon fluid are determined from the flocculation parameter resulting from a thermodynamic model applied to the calibrated compositional model of the hydrocarbon fluid. Thus, the adjusted decomposition of the hydrocarbon fluid makes it possible to determine the implementation of the flocculation, for example in order to determine a flocculation curve of the hydrocarbon fluid at a given temperature.

Furthermore, the invention relates to a process for determination or prediction of an asphaltene flocculation of a hydrocarbon fluid within a subterranean formation or within a fluid transportation pipe or within a conversion reactor (for example for a deasphalting process), in which the following stages are carried out:
a. conditions for asphaltene flocculation of the hydrocarbon fluid are determined by use of the process for determination of conditions for flocculation of asphaltenes according to any one of the alternative forms or of the combinations of alternative forms described above;
b. a pressure and a temperature of the hydrocarbon fluid within the subterranean formation or within the transportation pipe or within a conversion reactor are determined or predicted, by simulation or by measurement; and
c. a possible asphaltene flocculation of the hydrocarbon fluid is determined or predicted as a function of the determined flocculation conditions and of the determined or predicted pressure and of the determined or predicted temperature.

Thus, by virtue of this process, it is possible to determine a possible asphaltene flocculation during the exploitation of such a fluid, and it is also possible to simulate a possible asphaltene flocculation within a simulation of an exploitation of such a fluid. The exploitation of a hydrocarbon fluid can in particular concern the recovery of hydrocarbons within a subterranean formation, the transportation of hydrocarbons from the subterranean formation or the transportation of a petroleum fluid, the treatment of a hydrocarbon fluid within a conversion reactor (for example for a deasphalting process). Thus, the simulation concerned can be a simulation of hydrocarbon fluid transportation within a subterranean formation or a simulation of hydrocarbon fluid transportation within a transportation pipe or a reaction of the hydrocarbon fluid within a conversion reactor.

Advantageously, stages a) and c) can be carried out by a computer system, in particular a computer or a server.

Advantageously, the transportation pipe can be a pipe in a well for the production of hydrocarbons or a pipe of a line for the production of hydrocarbons.

Furthermore, when the process makes possible a simulation, stage b) can also be carried out by computer.

FIG. 4 illustrates, diagrammatically and without limitation, the stages of this process according to one embodiment. The stages already described for FIG. 2 are not described in detail again. The process comprises an additional stage of determination of prediction DET of a possible asphaltene flocculation AF, which depends on the flocculation conditions FC and on the operating conditions OPC, in other words on the temperature and pressure which are determined or predicted.

This figure does not represent the possible stage of measurement or of simulation of the operating conditions OPC.

Furthermore, the embodiment of FIG. 4 can be combined with the embodiment of FIG. 3.

In addition, the invention relates to a process for exploitation of a hydrocarbon fluid within a subterranean formation or within a fluid transportation pipe or within a conversion reactor, in which the following stages are carried out:
1. an asphaltene flocculation of the hydrocarbon fluid is determined or predicted by use of the process for determination or prediction of a flocculation according to any one of the alternative forms or of the combinations of alternative forms which are described above; and
2. the hydrocarbon fluid is exploited as a function of the possible asphaltene flocculation of the hydrocarbon fluid, in particular by modifying at least one of the temperature, the pressure, the composition of the hydrocarbon fluid and by adding a solvent in the case of asphaltene flocculation.

Thus, if a risk of asphaltene flocculation is determined or predicted, the process of exploitation of the hydrocarbon fluid can be adapted, and thus asphaltene flocculation can be avoided or at any rate the asphaltene flocculation can be limited. In this way, it is possible to ensure control of an exploitation of a hydrocarbon fluid. The exploitation of a hydrocarbon fluid can in particular concern the recovery of hydrocarbons within a subterranean formation, the transportation of hydrocarbons from the subterranean formation or the transportation of a petroleum fluid between two installations or two sites, a deasphalting process in a conversion reactor.

For this, it is possible in particular to at least one of increase, decrease the temperature of the fluid, and it is possible to at least one of increase or decrease the pressure of the fluid, adjust the composition of the fluid and to add a solvent.

FIG. 5 illustrates, diagrammatically and without limitation, the stages of this process according to one embodiment. The stages already described for FIG. 4 are not described in detail again. The process comprises an additional stage of exploitation EXP of the hydrocarbon fluid as a function of the possible asphaltene flocculation AF.

This figure does not represent the possible stage of measurement or of simulation of the operating conditions OPC.

Furthermore, the embodiment of FIG. 5 can be combined with the embodiment of FIG. 3.

### Applicational Example

The characteristics and advantages of the process according to the invention will become more clearly apparent on reading the applicational example below.

For this example, the aim is to determine the flocculation curve of a fluid considered and denoted Oil_9 in the following document: Sullivan, M., et al., 2020, A Fast Measurement of Asphaltene Onset Pressure, SPE Reservoir Evaluation & Engineering, 23 (03), 962-978.

The experimental data of this document, such as the saturation pressure and lower threshold pressure at three different temperatures, 75 °C, 100 °C and 125 °C, are used for the validation of the models according to the invention.

For this example, the stages of the process according to the invention are applied. In particular, the flocculation model is constructed by machine learning, starting from a training database which comprises 53 fluids. In addition, for this example, a compositional model is applied with a decomposition of the fluid into 10 constituents:
4 gases: N₂, H₂S, CO₂, CH₄;
1 light pseudocomponent in the C₂ to C₆ cut;
1 intermediate pseudocomponent in the C₇₊ to C₂₀ cut; and
4 heavy pseudocomponents, one per Saturates, Aromatics, Resins, Asphaltenes (SARA) fraction (on the C₂₀₊344 °C cut)

The calibration of the asphaltene constituent of the compositional model is carried out on the saturation pressures and the lower threshold pressures resulting from the flocculation model. In addition, the thermodynamic model used for this calculation can be the Peng-Robinson cubic equation of state coupled with the Abdoul-Péneloux mixing rule.

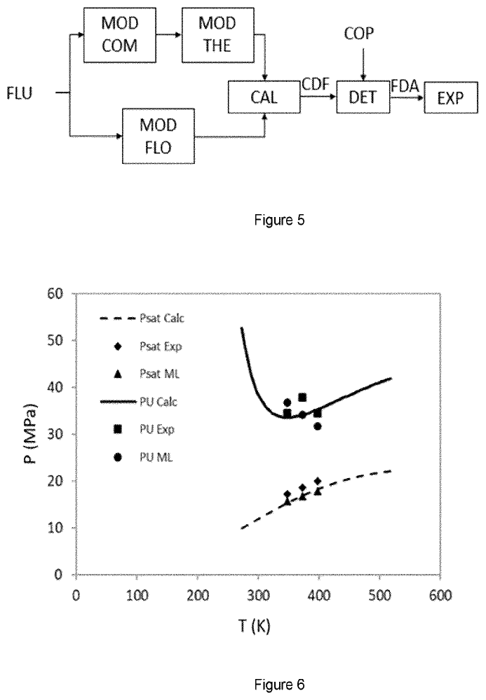

FIG. 6 illustrates, for this example, the pressures P in MPa as a function of the temperature T in K. In this figure, the index Calc indicates calculated (that is to say, obtained by the thermodynamic model and the calibrated compositional model), the index Exp indicates experimental (resulting from the cited document) and the index ML indicates obtained by the flocculation model of the process according to the invention. Pₛₐₜ denotes the saturation pressure and P_{U} denotes the lower threshold pressure. The calculated Calc pressure curves reproduce well experimental reference data, which makes possible the validation of the calibration carried out in the process according to the invention. These curves are generated by considering a wider temperature range in order to visualize the change in the key pressures (between 0 °C and 250 °C). In this figure, it is noticed that the pressures obtained by use of the flocculation model are consistent with the experimental data presented in the document (Sullivan et al., 2020). Thus, the flocculation model makes possible accuracy in the determination of the flocculation parameters.

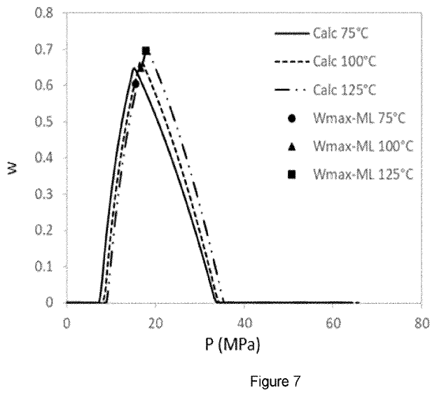

Subsequently, the flocculation curves are determined by use of the thermodynamic model. FIG. 7 illustrates, for this example, the amount of flocculated asphaltene W as a function of the pressure P in MPa for the three temperatures 75 °C, 100 °C and 125 °C. In this figure, Calc indicates calculated by use of the process according to the invention and Wmax-ML indicates the maximum amount of flocculated asphaltene determined by the flocculation model of the process according to the invention. The Wₘₐₓ points calculated by the flocculation model, which are not used in the calibration, are also in very good agreement with the flocculation curves. It should be noted that there are no asphaltene precipitation data in the paper (Sullivan et al., 2020) to experimentally validate the appearance of the whole of the flocculation curve. The results presented in this example thus make it possible to show the performance of the process according to the invention for the determination of the flocculation curve of an asphaltenic fluid in a scenario where the experimental data are absent.

1-10. (canceled)

11. A process for determination of conditions of asphaltene flocculation in a hydrocarbon fluid, comprising:
a) applying a flocculation model to the hydrocarbon fluid to determine at least one modeled flocculation parameter, the flocculation model relating a composition of a hydrocarbon fluid to at least one flocculation parameter;
b) constructing a compositional model of the hydrocarbon fluid, decomposing the hydrocarbon fluid into constituents, and determining at least one physical property of the hydrocarbon fluid by use of the compositional model;
c) applying a thermodynamic model to the at least one physical property to determine at least one flocculation parameter;
d) calibrating the compositional model of the hydrocarbon fluid by minimization of a difference between at least one flocculation parameter obtained from the thermodynamic model and the at least one modeled flocculation parameter obtained from the flocculation model; and
e) determining the conditions for asphaltene flocculation of the hydrocarbon fluid from the calibrated compositional model of the hydrocarbon fluid.

12. A process of constructing a flocculation model in accordance with claim 11, comprising:
i) constructing a training database of training fluids, wherein each training fluid is characterized by a composition and at least one flocculation parameter; and
ii) constructing the flocculation model by a machine learning method trained on the training database, the flocculation model relating the composition of the hydrocarbon fluid to the at least one flocculation parameter.

13. The process as claimed in claim 12, comprising experimentally obtaining the at least one flocculation parameter of the training fluid.

14. The process as claimed in claim 12, choosing each flocculation parameter from a lower threshold pressure, an upper threshold pressure, a saturation pressure or a maximum amount of flocculated asphaltene.

15. The process as claimed in claim 13, choosing each flocculation parameter from a lower threshold pressure, an upper threshold pressure, a saturation pressure or a maximum amount of flocculated asphaltene.

16. The process as claimed in claim 11, comprising basing compositional model on a representation in N constituents with N being between 6 and 15.

17. The process as claimed in claim 11, calibrating the compositional model with an iterative method.

18. The process as claimed in claim 11, wherein the thermodynamic model comprises an algorithm for calculating a three-phase liquid-liquid-vapor equilibrium of a fluid at a given temperature and at a given pressure.

19. The process as claimed in claim 11, comprising representing the flocculation conditions by a flocculation curve which relates an amount of flocculated asphaltene to pressure of the hydrocarbon fluid.

20. The process as claimed in claim 11, choosing at least one physical property from molar mass, critical temperature, critical pressure or an acentric factor of an constituents of the hydrocarbon fluid.

21. A process for determination or prediction of an asphaltene flocculation of a hydrocarbon fluid within a subterranean formation, a fluid transportation pipe or within a conversion reactor, comprising:
a) determining conditions for asphaltene flocculation of the hydrocarbon fluid in accordance with claim 11;
b) determining a pressure and a temperature of the hydrocarbon fluid within the subterranean formation, within the fluid transportation pipe or within the conversion reactor by measurement, prediction, or simulation; and
c) determining or predicting possible asphaltene flocculation of the hydrocarbon fluid as a function of the determined flocculation conditions, by determined or predicted pressure or by determined or predicted temperature.

22. A process for exploitation of a hydrocarbon fluid within at least one of a subterranean formation, within a fluid transportation pipe or within a conversion reactor, comprising:
a) determining predicted asphaltene flocculation of the hydrocarbon fluid by a process for determination or prediction of asphaltene flocculation as claimed in claim 21; and
b) exploiting the hydrocarbon fluid as a function of asphaltene flocculation of the hydrocarbon fluid.

23. A process in accordance with claim 22, comprising the hydrocarbon fluid by at least one of modifying at least one of temperature, pressure, and composition of the hydrocarbon fluid of asphaltene flocculation or by addition of a solvent to the hydrocarbon fluid.
